(19) United States
(12) Patent Application Publication
(10) Pub. No.: US 2012/0071692 A1
(43) Pub. Date: Mar. 22, 2012

(54) PROCESS FOR THE PREPARATION OF AROMATIC AMINES

(75) Inventors: SEBASTIAN AHRENS, Wiesloch (DE); Christoph Mueller, Mannheim (DE); Axel Paul, Lampertheim (DE); Martin Ernst, Heidelberg (DE); Nadja Pollmer, Bissersheim (DE); Johann-Peter Melder, Bohl-Iggelheim (DE); Thomas Heidemann, Viernheim (DE); Joachim-Thierry Anders, Dresden (DE); Bram Willem Hoffer, Bergen Op Zoom (NL)

(73) Assignee: BASF SE, Ludwigshafen (DE)

(21) Appl. No.: 13/234,328

(22) Filed: Sep. 16, 2011

Related U.S. Application Data
(60) Provisional application No. 61/383,754, filed on Sep. 17, 2010.

(30) Foreign Application Priority Data
Sep. 17, 2010 (EP) 10177390.1

(51) Int. Cl. C07C 209/18; B01J 21/06

(52) U.S. Cl. 564/402; 502/339

(57) ABSTRACT

A process for preparing an aromatic amine by reacting a corresponding aromatic alcohol with an aminating agent selected from the group consisting of ammonia, primary amines and secondary amines, in the presence of hydrogen and a catalyst molding, at a temperature of from 60-300°. The catalyst molding comprises Zr, Pd and Pt and has an annular tablet form with an external diameter in the range from 2-6 mm, a height in the range from 1-4 mm and an internal diameter of from 1-5 mm or a topologically equivalent form with the same volume. Catalyst moldings comprising Zr, Pd and Pt are also provided. The catalyst molding has an annular tablet form with an external diameter in the range from 3-6 mm, a height in the range from 1-4 mm and an internal diameter of from 2-5 mm or a topologically equivalent form.

The present application includes, by reference, the prior U.S. Application 61/383,754 submitted on Sep. 17, 2010.

The present invention relates to a process for the preparation of an aromatic amine by reacting a corresponding aromatic alcohol with an aminating agent in the presence of hydrogen and a catalyst molding which has a specific annular tablet form or a topologically equivalent form. Furthermore, the present invention relates to catalyst moldings with a specific annular tablet form or a topologically equivalent form.

Aromatic amines, such as 2,6-xylidine, are used inter alia as intermediates in the production of fuel additives (U.S. Pat. No. 3,275,554; DE-A-21 25039 and DE-A-36 11 230), surfactants, medicaments and crop protection compositions, curing agents for epoxy resins, catalysts for polyurethanes, intermediates for producing quaternary ammonium compounds, plasticizers, corrosion inhibitors, synthetic resins, ion exchangers, textile auxiliaries, dyes, vulcanization accelerators and/or emulsifiers.

U.S. Pat. No. 5,072,044 discloses the dehydrogenation of cyclohexylamines to the corresponding aromatic amines over Li-doped Pd catalysts. The reaction is carried out at high reaction temperatures of 360-380° C.

EP-A-701 995 describes a process for the preparation of aromatic amines from the corresponding cycloaliphatic amines in the presence of hydrogen and ammonia and over a bimetallic palladium/platinum catalyst. The cycloaliphatic amine must first be made available for this in another process. Here, Pd/Pt—ZrO₂ catalysts in the form of strands, inter alia, are described for the conversion of dimethylcyclohexylamine (DMCHA) to 2,6-xylidine.

EP-A-22751 describes the reaction of phenols in the presence of ammonia and hydrogen to give the corresponding cyclohexylamines in the presence of noble metal catalysts with clay earth or carbon as support material.

EP-A-53 819 discloses a process for the preparation of cycloaliphatic and/or aromatic amines from phenols. The catalyst system used comprises Ru, Rh or Pt on an aluminum oxide support.

EP-A-167 996 describes a process for the preparation of aromatic amines by reacting the corresponding phenol, optionally in the presence of the corresponding recycled cycloaliphatic amine, in the presence of ammonia and hydrogen over a noble metal catalyst at atmospheric pressure and in two reaction zones connected in series. Preference is given to aluminum-oxide-supported catalysts.

CA Abstract No. 132:336078 (CN-B-1 087970) relates to the synthesis of 2,6-dimethylaniline from 2,6-dimethylphenol at 180-200° C. over a specific Pd/Al₂O₃—MgO/Al₂O₃ catalyst.

WO 2006/136573 discloses a process for the continuous preparation of primary aromatic amines by reacting the corresponding aromatic alcohols with ammonia in the presence of hydrogen over heterogeneous catalysts, where the heterogeneous catalyst comprises palladium, platinum and zirconium.

WO 2007/077148 describes a process for the continuous preparation of a primary aromatic amine by reacting a corresponding cycloaliphatic alcohol with ammonia in the presence of a heterogeneous catalyst, wherein the catalytically active mass of the catalyst, prior to its reduction with hydrogen, comprises palladium, platinum and zirconium.

The object of the present invention was to find an improved economical process for the preparation of primary aromatic amines from phenols. In particular, the process should permit better yields, space-time yields (STY) and selectivities. Moreover, the formation of typical by-products, such as cyclohexanols, dicyclohexylamine, diphenylamines inter glia, which can only be separated off from the product of value with difficulty, should be reduced. Furthermore, it was an aim of the present invention to provide a catalyst with a relatively high activity which retains its activity over the longest possible period. The catalyst used should thus have improved service lives and have to be regenerated less often in order to be able to reduce shut-downs to change the catalyst in industrial operation.

The object of the present invention was achieved by a process for the preparation of an aromatic amine by reacting a corresponding aromatic alcohol with an aminating agent selected from the group consisting of ammonia, primary amines and secondary amines,

in the presence of hydrogen and

of a catalyst molding,

at a temperature of from 60 to 300°,

wherein the catalyst molding comprises Zr, Pd and Pt and has an annular tablet form with an external diameter in the range from 2 to 6 mm and a height in the range from 1 to 4 mm and an internal diameter of from 1 to 5 mm or a topologically equivalent form with the same volume.

In the process according to the invention, an aromatic alcohol is used which is reacted with an aminating agent in the presence of hydrogen and a catalyst molding to give a corresponding aromatic amine.

Aromatic alcohols that can be used are practically all alcohols with an aromatic OH function in which an OH group is bonded to an sp²-hybridized carbon atom of the aromatic ring. Besides carbon atoms, the aromatic ring can also have one or more heteroatoms, such as N, O or S. The alcohols can also carry substituents or comprise functional groups which behave inertly under the conditions of the hydrogenating amination, for example alkyl, alkoxy, alkenyloxy, alkylamino or dialkylamino groups, or else are optionally hydrogenated under the conditions of the hydrogenating amination, for example CC double or triple bonds. If the intention is to aminate polyhydric aromatic alcohols, then by controlling the reaction conditions it is possible to obtain preferably corresponding amino alcohols or polyaminated products.

Examples of aromatic alcohols which can be used in the process according to the invention are ortho-, meta- and para-cresol, ortho-ethylphenol, ortho-n-butylphenol, ortho-sec-butylphenol, 2,4-dimethylphenol, 2,6-dimethylphenol, 2,3,6-trimethylphenol, 2,4,6-trimethylphenol, 2-cyclohexylphenol, 2,6-dimethyl-3-cyclohexylphenol, 2,6-diethylphenol, 2,5-diisopropylphenol, 2-methyl-6-sec-butylphenol, 3-tert-butylphenol, 2,6-diisopropylphenol, 2,6-di-sec-butylphenol, 2,6-dicyclohexylphenol, alpha-naphthol, beta-naphthol, bisphenol A(=2,2-di(p-hydroxyphenyl)propane, hydroquinone, mono-, di-, tri- or tetraalkylhydroquinones, in particular hydroquinones substituted (independently of one another) with C₁₋₉-alkyl radicals, e.g. monomethylhydroquinone, tetramethylhydroquinone.

Preference is given to using aromatic alcohols of the formula (I)

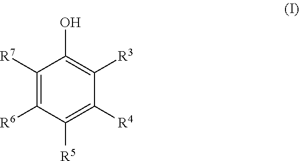

in which R³, R⁴, R⁵, R⁶ and R⁷ are in each case together or independently of one another

hydrogen,

C₁-C₁₂-alkyl, preferably C₁- to C₈-alkyl, particularly preferably C₁- to C₄-alkyl, such as methyl, ethyl, n-propyl, isopropyl, n-butyl, isobutyl, sec-butyl and tert-butyl, or

C₃-C₁₂-cycloalkyl, preferably C₅- to C₈-cycloalkyl, particularly preferably C₅- to C₆-cycloalkyl, such as cyclopentyl and cyclohexyl.

Particular preference is given to using alkyl-substituted phenols in the process according to the invention, where the substituents are preferably C₁₋₁₂-alkyl, particularly preferably C₁₋₉-alkyl and very particularly preferably C₁₋₄-alkyl.

It is also preferred that 1 to 3, preferably 1 to 2, of the substituents R³, R⁴, R⁵, R⁶ and R⁷ are corresponding alkyl substituents and the other substituents which are not alkyl are hydrogen.

Examples of preferred alkyl-substituted phenols are ortho-, meta- and para-cresol, ortho-ethylphenol, ortho-n-butylphenol, ortho-sec-butylphenol, 2,4-dimethylphenol, 2,6-dimethylphenol, 2,3,6-trimethylphenol, 2,4,6-trimethylphenol, 2-cyclohexylphenol, 2,6-dimethyl-3-cyclohexylphenol, 2,6-diethyiphenol, 2,5-diisopropylphenol, 2-methyl-6-sec-butylphenol, 3-tert-butylphenol, 2,6-di-sec-butylphenol or 2,6-dicyclohexylphenol.

In one preferred embodiment, 2,6-di(C₁₋₁₂-alkyl)phenols are used in the process according to the invention, preferably 2,6-di(C₁₋₈-alkyl)phenols and particularly preferably 2,6-di(C₁₋₄-alkyl)phenols.

Aromatic amines preferably prepared using the process according to the invention are 2,6-di(C₁₋₈-alkyl)anilines from the corresponding 2,6-di(C₁₋₈-alkyl)phenols. Examples are 2,6-dimethylaniline (2,6-xylidine), 2,6-diethylaniline, 2-methyl-6-ethylaniline, 2,6-diisopropylaniline, 2-isopropyl-6-methylaniline and 2-isopropyl-6-ethylaniline.

An aromatic amine particularly preferably prepared with the process according to the invention is 2,6-dimethylaniline (2,6-xylidine) by reacting 2,6-dimethylphenol.

The further starting material used in the process according to the invention is an aminating agent.

Aminating agents which can be used are either ammonia or primary or secondary amines, such as aliphatic or cycloaliphatic or aromatic amines.

The aminating agent is preferably a nitrogen compound of the formula II

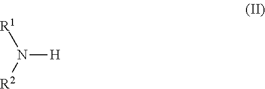

in which
R¹, R² are in each case together or independently of one another hydrogen (H),
   alkyl, such as C₁₋₁₂-alkyl, preferably C₁₋₈-alkyl, particularly preferably C₁₋₄-alkyl, in particular methyl, ethyl, n-propyl, isopropyl, n-butyl, isobutyl, sec-butyl and tert-butyl,
   cycloalkyl, such as C₃₋₁₂-cycloalkyl, preferably C₅- to C₈-cycloalkyl, particularly preferably C₅- to C₆-cycloalkyl, in particular cyclopentyl and cyclohexyl,
   alkoxyalkyl, such as C₂₋₃₀-alkoxyalkyl,
   dialkylaminoalkyl, such as C₃₋₃₀-dialkylaminoalkyl,
   aryl, such as C₆-C₁₄-aryl, such as phenyl, naphthyl or anthracenyl;
   aralkyl, such as C₇₋₂₀-aralkyl, preferably C₇₋₁₂-aralkyl, such as e.g. phenylmethyl, 1-phenylethyl, 2-phenylethyl, 1-phenylpropyl, 2-phenylpropyl or 3-phenylpropyl,
   or are together —(CH₂)ⱼ—X—(CH₂)ₖ—
   and
X is CH₂, CHR¹⁰ oxygen (O), sulfur (S) or NR¹⁰,
R¹⁰ is hydrogen (H), alkyl, preferably C₁₋₄-alkyl, alkylphenyl, preferably C₇₋₄₀-alkylphenyl,
j, k are an integer from 1 to 4.

Particularly preferably
R¹, R² are in each case together or independently of one another hydrogen (H),
   alkyl, such as C₁₋₁₂-alkyl, preferably C₁₋₈-alkyl, particularly preferably C₁₋₄-alkyl, in particular methyl, ethyl, n-propyl, isopropyl, n-butyl, isobutyl, sec-butyl and tert-butyl,
   cycloalkyl, such as C₃₋₁₂-cycloalkyl, preferably C₅- to C₈-cycloalkyl, particularly preferably C₅- to C₆-cycloalkyl, in particular cyclopentyl and cyclohexyl, or
   aryl, such as C₆-C₁₄-aryl, such as phenyl, naphthyl or anthracenyl.

The aforementioned substituents can also carry substituents or comprise functional groups which behave inertly under the conditions of the hydrogenating amination, for example alkyl, alkoxy, amino, alkylamino or dialkylamino groups, or else are optionally hydrogenated under the conditions of the hydrogenating amination, for example CC double or triple bonds.

The aminating agent is very particularly preferably selected from the group consisting of ammonia, monomethylamine, dimethylamine, monoethylamine, diethylamine, n-propylamine, di-n-propylamine, isopropylamine, diisopropylamine, isopropylethylamine, n-butylamine, di-n-butylamine, s-butylamine, di-s-butylamine, isobutylamine, n-pentylamine, s-pentylamine, isopentylamine, n-hexylamine, s-hexylamine, isohexylamine, cyclohexylamine, aniline, toluidine, piperidine, morpholine and pyrrolidine.

Especially preferred aminating agents are ammonia and monomethylamine and dimethylamine. Very particular preference is given to using ammonia as aminating agent.

As further feed material, hydrogen is used in the process according to the invention. The hydrogen is generally used in technical grade purity. The hydrogen can also be used in the form of a hydrogen-comprising gas, i.e. in admixtures with other inert gases, such as nitrogen, helium, neon, argon or carbon dioxide. Hydrogen-comprising gases which can be used are, for example, reformer offgases, refinery gases etc., if and so far as these gases do not comprise any catalyst poisons for the catalysts used, such as, for example, CO. However, preference is given to using pure hydrogen or essentially pure hydrogen in the process, for example hydrogen with a content of more than 99% by weight hydrogen, preferably more than 99.9% by weight hydrogen, particularly preferably more than 99.99% by weight hydrogen, in particular more than 99.999% by weight hydrogen.

The process according to the invention is carried out in the presence of a catalyst molding.

The catalyst molding is notable for the fact that the catalyst molding has an annular tablet form.

The external diameter DA of the annular tablet is in the range from 3 to 6 mm, preferably in the range from 4 to 6 mm and very particularly preferably in the range from 5 to 6 mm.

The internal diameter Dᵢ of the annular tablet is in the range from 2 to 5 mm, preferably in the range from 3 to 5 mm and very particularly preferably in the range from 3 to 4 mm.

The height h (or thickness) of the annular tablet is in the range from 1 to 4 mm, preferably in the range from 2 to 4 mm and very particularly preferably in the range from 2.5 to 3.5 mm.

It is also possible for the molding to have a geometry deviating from the annular tablet form which is topologically equivalent to or homomorphous with the annular tablet form. For example, the molding can have any desired geometry, such as elliptic, square, rectangular, triangular, rhombic, stellate etc. and be provided with a cavity or hollow (or hole) which can likewise have any desired geometry.

The volume of the topologically equivalent molding is selected such that the volume of the topologically equivalent molding is in the region of the volume of an annular tablet according to the invention (V=((D_{A}−Dᵢ)^{2·}π/4)·h).

The volume of the hollow or cavity of the topologically equivalent molding is accordingly selected such that the volume of the hollow or cavity is in the region of the volume of a hollow or cavity of an annular tablet according to the invention (V=(Dᵢ^{2·}π/4)·h).

In one preferred embodiment, the dimensions of the moldings, and of the topologically equivalent moldings are such that the largest diameter of the molding D_{A,max} is greater than the height of the molding at its highest point hₘₐₓ. Preferably, the ratio of D_{A,max} to hₘₐₓ is in the range from 1:0.1 to 1:0.95, particularly preferably in the range from 1:0.3 to 1:0.9 and particularly preferably in the range from 1:0.5 to 1:0.8.

If the molding has a topologically equivalent form deviating from the annular form, then it is further preferred for the hollow or the cavity to be configured such that the wall thickness of the molding at its thinnest point is preferably at least 0.5 mm, particularly preferably at least 1.0 mm and particularly preferably at least 1.2 mm. Preferably, the wall thickness of the topologically equivalent molding is preferably in the range from 0.5 to 4 mm, preferably in the range from 1 to 3 mm and very particularly preferably in the range from 1.2 to 2.5 mm. If the wall thickness is less than 0.5 mm, the molding is no longer so readily mechanically loadable, meaning that the molding can break. This can lead to blockages of the catalyst bed.

Furthermore, it is possible for the height h of the molding to vary within the range of the aforestated range from 1 to 4 mm, preferably in the range from 2 to 4 mm and very particularly preferably in the range from 2.5 to 3.5 mm. For example, the molding can have rounded edges and/or corners (torus form), meaning that the height from the edge of the catalyst molding inwards can increase and/or decrease.

Preferably, however, the molding has an annular tablet (or annular disk form) since such a form can be produced most simply and economically and, moreover, causes a low pressure drop in the catalyst bed.

The catalyst molding comprises a catalytically active mass and optionally further additives, such as shaping auxiliaries, support materials or further catalytically active constituents.

The details with regard to the composition (in % by weight) refer to the total mass of the catalyst molding after its last heat treatment (drying or calcination) and before treating the catalyst molding with hydrogen (reduction and/or activation).

In this form, the catalyst molding generally comprises less than 5% by weight of water, preferably less than 3.5% by weight of water and particularly preferably less than 1.5% by weight of water.

The catalyst molding preferably comprises 50% by weight and more of a catalytically active mass.

The catalytically active mass of the catalyst molding is defined as the sum of the masses of the oxygen-containing compounds of Zr, Pd and Pt after the last heat treatment of the manufactured catalyst molding and before the treatment (reduction and/or activation) with hydrogen.

Particularly preferably, the catalyst molding comprises 75% by weight and more, particularly preferably 90% by weight and more and very particularly preferably 94% by weight and more, of catalytically active mass.

In one preferred embodiment, the catalytically active mass of the catalyst molding used in the process according to the invention comprises

75 to 99.8% by weight, preferably 90 to 99.6% by weight, particularly preferably 94 to 99.2% by weight, of zirconium dioxide (ZrO₂),

0.1 to 12.5% by weight, preferably 0.2 to 5% by weight, particularly preferably 0.2 to 2.5% by weight and very particularly preferably 0.2-0.8% by weight, of oxygen-containing compounds of palladium and

0.1 to 12.5% by weight, preferably 0.2 to 5% by weight, particularly preferably 0.2 to 2.5% by weight and very particularly preferably 0.2-0.8% by weight, of oxygen-containing compounds of platinum.

Besides the catalytically active mass, the catalyst molding can comprise further additives, such as further catalytic constituents, shaping auxiliaries or support materials.

For example, as additive, the catalyst molding can comprise further catalytically active constituents.

Preferably, the further catalytically active constituents are elements of groups 1, 2, 13 to 15, and also IB to VIII B of the Periodic Table of the Elements, where the further catalytically active constituents, after the last heat treatment and before treatment with hydrogen (reduction and/or activation), are generally present, depending on their ability to be oxidized, either in the form of their element (oxidation state 0) or in the form of their oxygen-containing compounds.

The fraction of further catalytically active constituents in the additives, based on the catalyst molding, is preferably 0 to 50% by weight, preferably 0.01 to 25% by weight and particularly preferably 1 to 10% by weight.

In a very particularly preferred embodiment, the catalyst moldings essentially comprise no further catalytically active constituents.

The catalyst molding can furthermore comprise support materials.

Suitable support materials are, for example, carbon, such as graphite, carbon black and/or activated carbon, aluminum oxide (gamma, delta, theta, alpha, kappa, chi or mixtures thereof), silicon dioxide, zeolites, alumosilicates or mixtures thereof.

The fraction of support materials based on the catalyst molding is preferably 0 to 50% by weight, preferably 1 to 25% by weight and particularly preferably 3 to 10% by weight.

In a very particularly preferred embodiment, the catalyst moldings comprise essentially no further support materials.

The catalyst molding can comprise shaping auxiliaries as additives.

Preferred shaping auxiliaries are graphite or stearic acid.

The fraction of shaping auxiliaries based on the catalyst molding is preferably 0 to 20% by weight, preferably 1 to 15% by weight and particularly preferably 3 to 10% by weight.

In a very particularly preferred embodiment, the catalyst moldings comprise essentially no shaping auxiliaries.

To produce the catalyst moldings with the stated geometry and composition, various methods are possible.

In one preferred embodiment, a catalyst molding with a geometry according to the invention is produced which comprises Zr in the form of zirconium dioxide (ZrO₂) and which is then brought into contact with a solution which comprises soluble compounds of Pd and Pt (impregnation).

Zirconium dioxide can be used in the monoclinic or tetragonal modification. Preference is given to using a zirconium dioxide that is present to 60% by weight or more in the monoclinic modification, particularly preferably to 75% by weight or more in the monoclinic modification and very particularly preferably to 90% or more in the monoclinic modification.

Prior to the shaping, the zirconium dioxide is usually conditioned.

The conditioning can take place for example by adjusting zirconium dioxide to a certain particle size by grinding.

The adjustment of the particle sizes can take place by means of methods known to the person skilled in the art, such as grinding or spray-drying.

Preference is given to using zirconium dioxide with a particle size of from 0.1 μm to 0.1 mm, particularly preferably 0.5 μm to 500 μm and very particularly preferably 1 μm to 100 μm.

The conditioning can also include the mixing of zirconium dioxide with a shaping auxiliary, preferably graphite or stearic acid, particularly preferably graphite, or optionally further support materials.

If desired, prior to the shaping, solvents, pasting agents or pore formers can also be added to the conditioned zirconium dioxide.

Solvents and/or pasting agents can be added for example in order to modify the consistency of the conditioned zirconium dioxide. This may optionally be advantageous if the shaping is to take place by extrusion (see below).

Suitable solvents are e.g. acyclic or cyclic ethers having 2 to 12 carbon atoms, such as diethyl ether, di-n-propyl ether or isomers thereof, MTBE, THF, pyran, or lactones, such as gamma-butyrolactone, polyethers, such as monoglyme, diglyme etc., aromatic or aliphatic hydrocarbons, such as benzene, toluene, xylene, pentane, cyclopentane, hexane and petroleum ether, or mixtures thereof and particularly also N-methylpyrrolidone (NMP) or water or aqueous organic solvents or diluents of the type specified above. Both Brönsted acids and also Brönsted bases can be added to the water.

The fraction of solvents is preferably in the range from 0.5 to 80% by weight, further preferably in the range from 1 to 50% by weight, further preferably in the range from 1 to 40% by weight, and particularly preferably in the range from 1 to 30% by weight, in each case based on the mass of the conditioned zirconium dioxide used.

Pasting agents which can be used are all compounds suitable for this purpose. These are preferably organic, in particular hydrophilic, polymers, such as, for example, cellulose, cellulose derivatives, such as, for example, methylcellulose, starch, such as, for example, potato starch, polyacrylates, polymethacrylates, polyvinyl alcohol, polyvinylpyrrolidone (PVP), polyisobutene (PIB) or polytetrahydrofuran (PTHF).

The fraction of pasting agents is preferably in the range from 0.5 to 80% by weight, further preferably in the range from 1 to 50% by weight, further preferably in the range from 1 to 40% by weight, and particularly preferably in the range from 1 to 30% by weight, in each case based on the mass of the conditioned zirconium dioxide used.

Pore formers which can be used in the process according to the invention are all compounds which, with regard to the finished molding, provide a certain pore size, a certain pore size distribution and/or certain pore volumes.

As pore formers in the process according to the invention, preference is given to using polymers which can be dispersed, suspended and/or emulsified in water or in aqueous solvent mixtures. Preferred polymers here are polymeric vinyl compounds, such as, for example, polyalkylene oxides, such as polyethylene oxides, polystyrene, polyacrylates, polymethacrylates, polyolefins, polyamides and polyesters, carbohydrates, such as cellulose or cellulose derivatives, such as, for example, methylcellulose, or sugars or natural fibers. Further suitable pore formers are pulp or graphite.

Preference is also given to organic acidic compounds which can be removed by calcination. Mention is made here of carboxylic acids, in particular C₁₋₈-carboxylic acids, such as, for example, formic acid, oxalic acid and/or citric acid. It is likewise possible to use two or more of these acidic compounds.

If pore formers are used, then the fraction of pore formers is preferably in the range from 0.5 to 80% by weight, preferably in the range from 1 to 50% by weight and particularly preferably in the range from 1 to 30% by weight, in each case based on the mass of the conditioned zirconium dioxide used.

Should this be desired for the pore size distribution to be attained, it is also possible to use a mixture of two or more pore formers.

In one particularly preferred embodiment of the process according to the invention, the pore formers are removed to at least 90% by weight, as described below, by calcination to give the porous molding.

According to one preferred embodiment of the process according to the invention, moldings are obtained here which have pore volumes, determined in accordance with DIN 66134, in the region of at least 0.1 ml/g, preferably in the range from 0.1 to 0.5 ml/g and particularly preferably in the range from more than 0.15 ml/g to 0.35 ml/g. The specific surface area of the molding according to the invention, determined in accordance with DIN 66131, is preferably at least 30 m²/g and in particular at least 50 m²/g.

Before the shaping, the conditioned zirconium dioxide is homogenized with the optional further added constituents, e.g. for a period in the range from 10 to 180 minutes. For the homogenization, particular preference is given to using, inter alia, mixers, kneaders, edge mills or extruders.

The homogenization is preferably carried out at ambient temperature or at elevated temperature. If a solvent is added, however, then preference is given to working below the boiling point of the solvent. The mixture of conditioned zirconium dioxide and optional further additives and optional further added constituents is homogenized until a uniform, thoroughly mixed mass has formed.

Following the homogenization, the shaping of the conditioned zirconium dioxide generally takes place to give a molding with a geometry according to the invention.

The shaping takes place by customary methods, as described, for example, in Ullmann's Encyclopedia of Industrial Chemistry, “Heterogeneous Catalysis and Solid Catalysts”, chapter 5.2.2. (DOI: 10.1002/14356007.a05_{—}313.pub2) or in James T. Richardson, “Principles of Catalyst Development (Perspectives on Individual Differences)”, Springer, Berlin, 1989, chapter 6.8, or in B. Stiles, T. A Koch, “Catalyst Manufacture”, M. Dekker, New York, 1995, chapter 9.

Here, the shaping preferably takes place by compaction or compression of the conditioned zirconium dioxide in molding molds with a geometry according to the invention, e.g. by pelleting or tableting.

The shaping can also take place by extrusion of the conditioned zirconium dioxide with suitable profile dies. For the extrusion, solvents and/or pasting agents are generally also added to the conditioned zirconium dioxide so that the conditioned zirconium dioxide has the correct consistency for further processing.

During the production of the moldings according to the invention by extrusion, after the shaping process, a heating step(=heat treatment) generally takes place, in particular a drying and/or calcination step, during which the molding is usually heat-treated at relatively high temperatures. As a rule, the drying and/or calcination step is carried out at temperatures of from 80 to 600°, preferably 120 to 450° C., particularly preferably 350 to 450° C.

If the production of the moldings according to the invention takes place by tableting, then as a rule no heating step preferably takes place after the shaping and before the impregnation.

The impregnation of the moldings with Pd and Pt compounds can take place by customary methods (A. B. Stiles, Catalyst Manufacture—Laboratory and Commercial Preparations, Marcel Dekker, New York, 1983), for example by applying soluble compounds of Pd and Pt or of the further catalytically active constituents in one or more impregnation stages.

Suitable soluble compounds are generally water-soluble metal salts, such as the nitrates, acetates or chlorides of Pd and Pt or of the further catalytically active constituents. The impregnation can also take place with other suitable soluble compounds of the corresponding elements.

Afterwards, the impregnated molding is generally dried and calcined.

The drying usually takes place at temperatures of from 80 to 200° C., preferably 100 to 150° C.

The calcination is generally carried out at temperatures of from 300 to 600° C., preferably 350 to 500° C., particularly preferably 380 to 480° C.

The impregnation can also take place by the so-called “incipient wetness method”, in which the catalyst molding is wetted with the impregnation solution according to its water absorption capacity at most to saturation. The impregnation can, however, also take place in supernatant solution.

In the case of multistage impregnation methods, it is expedient to carry out drying and optionally calcination between individual impregnation steps. The multistage impregnation can advantageously be used when the molding is to be supplied with soluble compounds in a relatively large amount.

To apply two or more components to the molding, the impregnation can for example take place simultaneously with all soluble compounds or in any desired order of the soluble compounds in succession.

After the calcination, the catalyst moldings obtained by impregnation comprise the components of the catalytically active mass in the form of a mixture of their oxygen-containing compounds, i.e. in particular as oxides, mixed oxides and/or hydrides. The catalyst moldings produced in this way can be stored and handled as such.

The catalyst moldings which are used in the process according to the invention are obtained by reducing and/or activating the catalyst moldings which have been produced as described above by impregnation after the final heating step (calcination).

Here, the activation or reduction of the catalyst molding can take place directly within the reactor under the conditions of the process according to the invention.

In one preferred embodiment, the reduction and/or activation of the catalyst molding takes place prior to its use in the process according to the invention (so-called pre-reduction).

The reduction of the dry catalyst molding can be carried out at elevated temperature in an agitated or non-agitated reducing oven.

The reducing agents used are usually hydrogen or a hydrogen-comprising gas.

The hydrogen is generally used in technical grade purity. The hydrogen can also be used in the form of a hydrogen-comprising gas, i.e. in admixtures with other inert gases, such as nitrogen, helium, neon, argon or carbon dioxide. The stream of hydrogen can also be returned to the reduction as recycled gas, optionally mixed with fresh hydrogen and optionally after removing water by condensation.

The reduction of the catalyst molding preferably takes place in a reactor in which the catalyst moldings are arranged in the form of a fixed bed. The reduction of the catalyst molding takes place particularly preferably in the same reactor in which the subsequent reaction of aromatic alcohol with aminating agent takes place.

The reduction of the catalyst molding generally takes place at reducing temperatures of from 50 to 600° C., in particular from 100 to 500° C., particularly preferably from 150 to 450° C.

The hydrogen partial pressure is generally from 0.1 to 300 bar, in particular from 0.1 to 100 bar, particularly preferably from 0.1 to 10 bar, the stated pressures referring here and below to the absolute measured pressure.

The reduction time is preferably 1 to 48 hours, and particularly preferably 5 to 15 hours. During the reduction, a solvent can be introduced in order to remove any water of reaction that is formed and/or in order to be able for example to heat the reactor more quickly and/or to be able to better dissipate the heat during the reduction. The solvent can also be introduced here in a supercritical manner.

Suitable solvents which can be used are the solvents described above. Preferred solvents are water; ethers such as methyl tert-butyl ether, ethyl tert-butyl ether, dioxane or tetrahydrofuran. Particular preference is given to water or tetrahydrofuran. Suitable mixtures are likewise suitable as suitable solvents.

The activated or reduced catalyst molding can be handled after the reduction under inert conditions. Preferably, the activated catalyst molding can be handled and stored under an inert gas such as nitrogen or is used under an inert liquid, for example an alcohol, water, the product or starting material of the particular reaction for which the catalyst molding is used. If applicable, the catalyst molding then has to be freed from the inert liquid prior to the start of the actual reaction.

Storage of the catalyst molding under inert substances permits uncomplicated and nonhazardous handling and storage of the catalyst molding.

After the reduction, however, the catalyst molding can also be brought into contact with an oxygen-comprising gas stream such as air or a mixture of air and nitrogen. This gives a passivated catalyst molding. The passivated catalyst molding generally has a protective oxide layer. This protective oxide layer makes it easier to handle and store the catalyst molding, meaning that, for example, integration of the passivated catalyst molding into the reactor is simplified. Passivated catalyst moldings are generally activated and/or reduced again as described above prior to the start of the reaction.

The aromatic amines are prepared by reacting the corresponding aromatic alcohol with an aminating agent in the presence of hydrogen and a catalyst molding at a temperature of from 60 to 300°, preferably in the gas phase. However, the reaction can also take place in the liquid phase.

For the synthesis in the gas phase, the aromatic alcohol used is evaporated in a targeted manner, preferably in a stream of recycled gas, and fed to the reactor in gaseous form. The recycled gas serves firstly for evaporation of the aromatic alcohol used and secondly as reactant for the amination.

In the recycled-gas procedure, the starting materials (aromatic alcohol, hydrogen and aminating agent) are evaporated in a stream of recycled gas and fed to the reactor in gaseous form.

The starting materials (aromatic alcohol and aminating agent) can also be evaporated as aqueous solutions and be supplied to the catalyst bed with the stream of recycled gas.

Preferred reactors are tubular reactors. Examples of suitable reactors with stream of recycled gas can be found in Ullmann's Encyclopedia of Industrial Chemistry, 5th Ed., Vol. B 4, pages 199-238, “Fixed-Bed Reactors”.

Alternatively, the reaction advantageously takes place in a tube-bundle reactor or in a mono-stream plant.

In a monostream plant, the tubular reactor in which the reaction takes place can consist of a serial connection of a plurality of, preferably two or three, individual tubular reactors. Optionally, an intermediate introduction of feed (comprising the starting material and/or ammonia and/or H₂) and/or recycled gas and/or reactor discharge from a downstream reactor is possible here in an advantageous manner.

With regard to the alcoholic hydroxyl group to be aminated, the aminating agent can be used in stoichiometric, substoichiometric or superstoichiometric amounts.

The aminating agent is generally used with a 1.5 to 250-fold, preferably 2 to 100-fold, in particular 2 to 10-fold molar excess per mole of alcoholic hydroxyl group to be reacted. Higher excesses of aminating agent are possible.

Preference is given to an offgas amount of from 5 to 800 cubic meters/h (STP), in particular 20 to 300 cubic meters/h (STP).

The process according to the invention is usually carried out at 0.1 to 40 MPa (1 to 400 bar), preferably 0.1 to 10 MPa, particularly preferably 0.1 to 0.5 MPa.

The temperatures are generally 60 to 300° C., particularly 100 to 290° C., preferably 120 to 280° C., particularly preferably 160 to 270° C.

In one particularly preferred embodiment, the process is carried out in a plant of 2 reactors connected in series, in particular tube-bundle reactors, or in a reactor with two temperature zones, it being preferred for the reactor temperature in the second reactor or in the first temperature zone to be in the range from 5 to 50°, preferably in the range from 10 to 40° C. and particularly preferably in the range from 15 to 35° C., above the temperature of the first reactor or of the first temperature zone.

The pressure in the reaction vessel, which arises from the sum of the partial pressures of the aminating agent, of the aromatic alcohol and of the reaction products formed, and optionally of the co-used solvent at the stated temperatures, is expediently increased to the desired reaction pressure by injecting hydrogen.

The catalyst hourly space velocity is generally in the range from 0.01 to 2, preferably 0.05 to 0.5 kg, of aromatic alcohol per liter of catalyst (bed volume) an hour.

Here, the flow to the fixed catalyst bed may be either from above or from below. The required gas stream is preferably obtained by a recycled-gas method.

The amount of recycled gas is preferably in the range from 40 to 2000 m³ (at operating pressure)/[m³ of catalyst (bed volume)·h], in particular in the range from 700 to 1700 m³ (at operating pressure)/[m³ of catalyst (bed volume)·h].

The recycled gas preferably comprises at least 10, particularly 50 to 100, very particularly 60 to 90, % by volume of H₂.

The hydrogen is generally fed to the reaction in an amount of from 5 to 400 I, preferably in an amount of from 5 to 100 I, per mole of aromatic alcohol component, the liters stated in each case having been converted to standard conditions (STP).

In the case of continuous operation in the gas phase, the excess aminating agent can be recycled together with the hydrogen.

The water of reaction formed in the course of the reaction (in each case one mol per mole of reacted alcohol group) generally does not have a disruptive effect on the degree of conversion, the reaction rate, the selectivity and the catalyst service life, and is therefore expediently not removed therefrom until the work-up of the reaction product, e.g. by distillation.

After the reaction discharge has been expediently decompressed, the excess hydrogen and any excess aminating agent present are removed therefrom and the resulting crude reaction product is purified, e.g. by a fractional rectification. The excess aminating agent and the hydrogen are advantageously returned to the reaction zone. The same applies to any incompletely converted aromatic alcohol.

The pure products in each case can be obtained from the crude products by rectification by the known methods. The pure products are produced as azeotropes with water or can be dewatered in accordance with the patent applications EP-A-1 312 599 and EP-A-1 312 600 by a liquid-liquid extraction with concentrated sodium hydroxide solution. This dewatering can take place before or after the purification by distillation. A distillative dewatering in the presence of an entrainer by known methods is also possible.

If the crude product or the aromatic amine in the crude product is barely miscible or immiscible with water, a dewatering by separating the organic and the aqueous phase using known methods is also possible. According to the procedure taught in EP-A-1 312 599 and in EP-A-1 312 600, in one step, one or more low-boiling fractions can be separated off from the amine-containing mixture by distillation in the separated organic phase. In a further step, it is possible to separate off one or more high-boiling fractions from the amine-containing mixture by distillation. In a subsequent distillation step, the essentially anhydrous amine can be obtained in pure form from the mixture as bottom take-off or side take-off of the column, and, if desired, is subjected to a further purification or separation.

These individual steps for purifying the aromatic amine can if desired also be carried out in a single column batchwise or continuously, it being possible to separate off the low-boiling components via the top take-off and/or the side take-off of the rectification section of the column, to separate off the high-boiling fractions via the bottom take-off of the distillation column and to separate off the pure amine via the side take-off in the stripping section of the column.

In one particularly preferred variant, a dividing wall column is used as continuous distillation column.

Unreacted starting materials and any suitable by-products which are formed can be returned to the synthesis. Unreacted starting materials can be streamed again in the recycled-gas stream over the catalyst bed in batchwise or continuous mode following condensation of the products in the separator.

The catalyst moldings according to the invention permit an improved economical process for the preparation of primary aromatic amines from phenols. In particular, better yields, space-time yields (STY) and selectivities are achieved with such a process. Moreover, the formation of typical by-products, such as cyclohexanols, dicyclohexylamine, diphenylamines, inter alia, which can only be separated off from the product of value with difficulty, is reduced. Furthermore, a catalyst with a higher activity is provided which maintains its activity over the longest possible period. The catalyst used leads to an improvement in service lives and thus permits less frequent regenerations, as a result of which shut-downs for changing the catalyst in industrial operation can be reduced.

The invention is illustrated by the examples below.

### EXAMPLES

Preparation of the Catalyst:

### Molding A (Comparative Example)

1.5 kg of ZrO₂ powder are mixed with 5% graphite and then compressed to give annular tablets measuring 7(D_{A′)}×3(Dᵢ)×3 (h) mm. The compacting pressure of the tablet press is adjusted such that a water absorption of 0.18-0.28 ml/g is obtained for the prepared tablets. The precise water absorption of the annular tablets is determined and the concentration of the impregnation solutions for an impregnation to water absorption with 0.46% PdO and 0.43% PtO is calculated. The annular tablets are then impregnated in an impregnating drum with a solution of palladium nitrate and platinum nitrate, such that the solution is sprayed onto the annular tablets. The rings are then dried at 120° C. for 4 h and then calcined at 520° C. for 2 h. The catalyst molding produced in this way comprised 0.46% by weight of PdO (calculated as metal in oxidation state II) and 0.43% by weight of PtO (calculated as metal in oxidation state II) based on ZrO₂.

### Molding B (According to the Invention)

1.5 kg of ZrO₂ powder are mixed with 5% graphite and then compacted to give annular tablets measuring 5.5(D_{A})×3(Dᵢ)×3 (h) mm. The compacting pressure of the tablet press is adjusted so that a water absorption of 0.18-0.28 ml/g is obtained for the prepared tablets. The precise water absorption of the annular tablets is determined and the concentration of the impregnation solutions for an impregnation to water absorption with 0.46% PdO and 0.43% PtO is calculated. The annular tablets are then impregnated in an impregnating drum with a solution of palladium nitrate and platinum nitrate, such that the solution is sprayed onto the annular tablets. The rings are then dried at 120° C. for 4 h and then calcined at 520° C. for 2 h. The catalyst molding produced in this way then comprises 0.46% by weight of PdO (calculated as metal in oxidation state II) and 0.43% by weight of PtO (calculated as metal in oxidation state II) based on ZrO₂.

Testing of the Catalysts:

The experiments were carried out in a continuously operated gas phase apparatus with oil-heated reactor (30×2×750 mm) at atmospheric pressure. For this, molten 2,6-dimethylphenol (DMP) was combined, prior to being inserted into the reactor, with hydrogen and ammonia and, after a mixing stretch, conveyed to the upper part of the reactor. The upper part of the reactor was filled with 250 ml of V2A steel rings (D=3 mm), below which was located the corresponding catalyst molding (A or B) (100 ml), under which in turn was located 50 ml of V2A steel rings (D=6 mm). After leaving the reactor, the reactor discharge was passed to a separator, in which the liquid discharge was collected.

Table 1 lists the results of the rate-determining first step of the xylidine preparation. The oven temperature in each case was adjusted to 200° C. The molar ratio of ammonia to hydrogen was kept at 1.55:1, the molar ratio of ammonia to 2,6-dimethylphenol (DMP) at 6.60:1. In the first time interval of the experiments, the catalyst hourly space velocity was adjusted to 0.153 kg/I_{cat.}h (0-950 h), then the hourly space velocity was increased in stages (0.176 kg/I_{cat.}h; 950-1150 h and 0.204 kg/I_{cat.}h; 1150-1330 h).

The results show that particularly the catalyst with the 5.5×3×3 mm molding is very active and is only very slowly deactivated; even after 1330 h, the residual DMP content is still less than 2%, whereas in the case of the otherwise identical catalyst with 7×3×3 mm molding, 6.3% residual DMP are found. The fraction of low-boiling by-products is also lower with the catalyst according to the invention. With the catalyst moldings according to the invention it is possible to achieve a higher conversion of 2,6-DMP. Moreover, the yield of products of value (2,6-xylidine and DMCH-a, DMCH-one and DMCH-ol) is increased and the amount of undesired low-boiling components is lower.

TABLE 1

| | | Hourly space | | | Low-boiling | DMCH -a, | |
|---|---|---|---|---|---|---|---|
| | | velocity | Run time | 2,6-DMP | components | -one, -ol | 2,6-Xylidine |
| Charge | Catalyst | [symbol] | [h] | [GC area %] | [GC area %] | [GC area %] | [GC area %] |
| 1 | 7 × 3 × 3 | 0.153 | 950 | 2.1 | 3.4 | 38.6 | 55.1 |
| 2 | 7 × 3 × 3 | 0.176 | 1150 | 3.3 | 3.2 | 39.7 | 53.7 |
| 3 | 7 × 3 × 3 | 0.204 | 1330 | 6.3 | 2.7 | 39.6 | 51.3 |
| 4 | 5.5 × 3 × 3 | 0.153 | 950 | 0.5 | 3.0 | 45.4 | 51.0 |
| 5 | 5.5 × 3 × 3 | 0.176 | 1150 | 1.0 | 2.4 | 45.9 | 50.6 |
| 6 | 5.5 × 3 × 3 | 0.204 | 1330 | 1.7 | 2.1 | 47.0 | 49.2 |

2,6-DMP = 2,6-Dimethylphenol
DMCH-a = 2,6-Dimethylcyclohexylamine
DMCH-one = 2,6-Dimethylcyclohexanone
DMCH-ol = 2,6-Dimethylcyclohexanol [symbol]
indicates data missing or illegible when filed

The aforementioned contents in % by weight are determined by gas chromatography as follows:

Separating column: DB WAX (polyethylene glycol)

Length (m): 30

Film thickness (μm): 0.5

Internal diameter (mm): 0.25

Carrier gas: Helium

Preliminary pressure (bar): 1.0

Split (ml/min): 100

Septum flushing (ml/min): 4

Oven temperature (° C.): 80

Preheating time (min): 3

Rate (° C./min): 5

Oven temperature (° C.): 240

Post-heating time (min): 30

Injector temperature (° C.): 250

Detector temperature (° C.): 260

Injection: HP 7673-Autosampler

Injection volume (microliters): 0.2

Detector type: FID

GC method: GC area % method

1-12. (canceled)

13. A process for the preparation of an aromatic amine by reacting a corresponding aromatic alcohol with an aminating agent selected from the group consisting of ammonia, primary amines and secondary amines,
in the presence of hydrogen and
a catalyst molding,
at a temperature of from 60 to 300°,
wherein the catalyst molding comprises Zr, Pd and Pt and an annular tablet form with an external diameter in the range from 2 to 6 mm and a height in the range from 1 to 4 mm and an internal diameter of from 1 to 5 mm or a topologically equivalent form with the same volume.

14. The process according to claim 13, wherein the catalyst molding comprises an active mass which, prior to reduction of the active mass with hydrogen, comprises
75 to 99.8% by weight of oxygen-containing compounds of zirconium,
0.01 to 12.5% by weight of oxygen-containing compounds of palladium and
0.01 to 12.5% by weight of oxygen-containing compounds of platinum.

15. The process according to claim 14, wherein the catalyst molding comprises 50% by weight and more of active mass, based on the catalyst molding.

16. The process according to claim 13, wherein the catalyst molding optionally comprises 0 to 50% by weight of one or more additives.

17. The process according to claim 16, wherein the additive is graphite.

18. The process according to claim 13, wherein the aromatic alcohol used is a phenol of the formula (I) 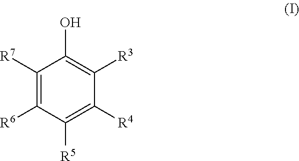
in which R³, R⁴, R⁵, R⁶ and R⁷ are in each case together or independently of one another hydrogen (H), C₁-C₁₂₋alkyl, or C₃-C₁₂-cycloalkyl.

19. The process according to claim 18, wherein the phenol used is 2,6-dimethylphenol.

20. The process according to claim 13, wherein the aminating agent is an aminating agent of the formula (II), 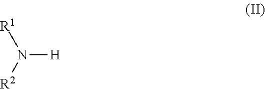
in which R¹, R² are in each case together or independently of one another hydrogen (H), alkyl, cycloalkyl, alkoxyalkyl, dialkylaminoalkyl, aryl, aralkyl, or together —(CH₂)ⱼ—X—(CH₂)ₖ— and X is CH₂, CHR¹⁰ oxygen (O), sulfur (S) or NR¹⁰ and R¹⁰ is hydrogen (H), alkyl or alkylphenyl and j, k are an integer from 1 to 5.

21. A catalyst molding comprising Zr, Pd and Pt, wherein the catalyst molding has an annular tablet form with an external diameter in the range from 3 to 6 mm and a height in the range from 1 to 4 mm and an internal diameter of from 2 to 5 mm or a topologically equivalent form.

22. The catalyst molding according to claim 21, wherein the catalyst molding comprises an active mass which, prior to the reduction of the active mass with hydrogen, comprises
75 to 99.8% by weight of zirconium dioxide (ZrO₂),
0.01 to 12.5% by weight of oxygen-containing compounds of palladium and
0.01 to 12.5% by weight of oxygen-containing compounds of platinum.

23. The catalyst molding according to claim 21, wherein the catalyst molding has 50% by weight and more of active mass and optionally comprises 0 to 50% by weight of additives.

24. The catalyst molding according to claim 21, wherein firstly a catalyst molding with the geometry according to the invention is produced which comprises Zr in the form of zirconium dioxide (ZrO₂) and which is then brought into contact with a solution which comprises soluble compounds of Pd and Pt (impregnation).
